(19) United States
(12) Patent Application Publication
(10) Pub. No.: US 2025/0213635 A1
(43) Pub. Date: Jul. 3, 2025

(54) USE OF A COMPOSITION FOR MANAGING POSTPRANDIAL GLUCOSE RESPONSE AND ASSOCIATED DISORDERS

(71) Applicant: SOCIETE DES PRODUITS NESTLE S.A., Vevey (CH)

(72) Inventors: NORMAN MOULLAN, St-Prex (CH); CHRISTIAN DARIMONT-NICOLAU, Lausanne (CH); TZE WAN CHERLYN DING, Singapore (SG); QU FU, Beijing (CN)

(21) Appl. No.: 18/843,065

(22) PCT Filed: Feb. 22, 2023
(86) PCT No.: PCT/EP2023/054356

§ 371 Date: Aug. 30, 2024

Related U.S. Application Data
(60) Provisional application No. 63/315,752, filed on Mar. 2, 2022.

(51) Int. Cl. A61K 36/605; A61K 31/201; A61K 31/202; A61K 31/445; A61K 31/733

(52) U.S. Cl. CPC A61K 36/605; A61K 31/201; A61K 31/202; A61K 31/445; A61K 31/733

(57) ABSTRACT

The present invention generally relates to the use of a composition comprising mulberry leaf extract, a source of fiber and a source of unsaturated fatty acids to decrease postprandial glucose (PPG) response. More particularly, it relates to the use of such composition, administered with a meal, to decrease postprandial glucose (PPG) response. The invention also relates to a composition comprising a mulberry leaf extract, a source of fiber and a source of unsaturated fatty acids, administered with a meal, for use in the treatment and/or prevention of a disorder linked to postprandial glucose and/or postprandial insulin in a subject.

The present invention generally relates to the use of a composition comprising mulberry leaf extract, a source of fiber and a source of unsaturated fatty acids to decrease postprandial glucose (PPG) response. More particularly, it relates to the use of such composition, administered with a meal, to decrease postprandial glucose (PPG) response. The invention also relates to a composition comprising a mulberry leaf extract, a source of fiber and a source of unsaturated fatty acids, administered with a meal, for use in the treatment and/or prevention of a disorder linked to postprandial glucose and/or postprandial insulin in a subject.

### BACKGROUND

Diabetes is a metabolic condition characterized primarily by high blood glucose levels that results from the body's inability to make or use insulin. Hyperglycemia can lead to numerous clinical complications including blindness, limb amputations, heart attack or stroke.

The most common types of diabetes are insulin-dependent diabetes (Type-1 diabetes T1D) and non-insulin-dependent diabetes (Type-2 diabetes T2D). T2D is by far the most abundant type, and the increase in Type-2 diabetes (T2D) is mainly driven by increasing obesity rates.

Additionally, pre-diabetic conditions, defined as having a blood glucose higher than normal but not high enough to be diagnosed as diabetic, are contributing significantly to the strong rise of the diabetic population.

Insulin resistance (a low insulin sensitivity) occurs also in pregnant subjects. This is due to hormonal changes that help to ensure the transfer of nutrients from the pregnant subject to the fetus. As described above, in response to insulin resistance the pancreas may secrete more insulin to compensate. These subjects are considered as having an impaired glucose tolerance (hereinafter IGT). Eventually the pancreas may fail to keep up with the body's increased need for insulin, leading to type-2 diabetes. Any degree of glucose intolerance with onset or first recognition during pregnancy is referred to as Gestational Diabetes Mellitus (GDM).

The pathophysiology of the development of Type-2 diabetes is complex and multifactorial. Obesity, sedentary life style, and/or increased age may lead to insulin resistance and to increased circulating insulin concentrations over time. At some point, a loss of control of blood glucose begins to emerge, resulting in impaired glucose tolerance (IGT) or impaired fasting glucose (IFG) and may ultimately result in Type-2 diabetes. Therefore, IGT and IFG refer to metabolic states intermediate between normal glucose homeostasis and diabetes.

A further test, the oral glucose tolerance test (OGTT), may be performed to assess whether the patient is diabetic or has IGT. The OGTT consists of a glucose drink containing 75 g of glucose. The patient's blood sugar level is measured at one and two hours following administration of the drink.

Glucose is an essential nutrient for the human body, so its circulating levels must be carefully maintained constant in order to supply adequate amounts to peripheral tissues. The liver plays a central role in glucose homeostasis by balancing uptake and storage of glucose via glycogenesis and its release via glycogenolysis and gluconeogenesis. An impairment of glucose homeostasis is a typical feature of Type-2 diabetes. Patients with Type-2 diabetes exhibit increased hepatic glucose production (HGP), which is identified as the main cause of fasting hyperglycemia and is associated with a reduced plasma glucose clearance (Gastaldelli A, et al., Diabetes 2000; 49:1367-1373), and also a 25-45% reduced synthesis of glycogen compared with non-diabetic subjects (Roden M, et al., Best Pract Res Clin Endocrinol Metab. 2003; 17:365-83).

Optimal glycemic control is fundamental to the management of diabetes. Both fasting plasma glucose (FPG) and postprandial plasma glucose (PPG) levels correlate with the risk of complications and contribute to the measured glycated hemoglobin (A1C) value. A1C levels >7.0% are associated with a significantly increase risk of both microvascular and cardiovascular (CV) complications. PPG is an important component of overall hyperglycemia and may be the predominant component in patients who are closer to A1C goal and in older adults.

PPG are determined by several factors, such as the total caloric value of a meal, macronutrient composition, and carbohydrate quality (e.g., glycemic index/load), all of which may be monitored and controlled. However, multiple other factors involved in diseases such as T2D are more complex because they cannot be controlled and are variable between individuals. These include gastric emptying rate, intestinal absorption rate, enteroendocrine incretin secretion, incretin sensitivity, pancreatic beta-cell insulin secretory function, hepatic glucose production, glucose effectiveness, glucose uptake in all tissues (especially brain, adipose, liver, and muscle), insulin sensitivity, and renal glucose reabsorption.

Limiting blood glucose peaks after a meal in diabetic subjects constitutes an important target of the overall glycemic control strategy. Uncontrolled PPG is common in diabetes. It contributes to overall hyperglycemia and is associated with poor outcomes. Treatment options that specifically target PPG are, therefore, critical components to achieving and sustaining glycemic control in patients with Type-1 diabetes (T1D) and Type-2 diabetes (T2D), and might prevent pre-diabetic subjects to advance to a diagnosed diabetic condition.

Evidences are revealing the importance of PPG and post-prandial insulin (PPI) in both the management and prevention of type 2 diabetes. PPG was shown to be the main contributor to total glucose fluctuations in T2D patients with HbA1c<8%, i.e. well controlled diabetes or prediabetes patients. Postprandialhyperinsulemia has been shown to be a marker of prediabetes and metabolic syndrome (M. Shanik et al., Diabetes Care 2008 February; 31(Supplement 2): S262-S268.). Prediabetes state is rapidly increasing worldwide and is mainly associated with age and BMI. Controlling PPG and/or PPI response in the overweight and obese population, also at risk for T2D, appears to be key for preventing this disease.

Actual treatments for T2D comprise several classes of drugs, which can be used alone or in combination with insulin.

Alpha-glucosidase inhibitors slow the digestion of carbohydrates, delay glucose absorption, and reduce the increase in blood glucose after a meal. Common side effects include abdominal pain, diarrhea, and flatulence. The antidiabetic drug Acarbose is an alpha-glucosidase inhibitor. However, gastrointestinal side-effects have been reported, in particular flatulence and diarrhea, due to the fact that it also inhibits alpha-amylase activity.

Carbohydrates are important nutrient of the human diet and the main dietary component affecting glycaemia. The amount and type of carbohydrate have an effect on both postprandial glycaemia and insulinaemia. Postprandial glucose, together with related hyperinsulinemia and lipidaemia, has been implicated in the development of chronic metabolic diseases like obesity, type 2 diabetes mellitus (T2DM) and cardiovascular disease (CVD). A meta-analysis performed on 24 prospective cohort studies has demonstrated a strong and significant lower risk to develop T2D in persons who consume lower Glycaemic load diet.

Mulberry leaves have been used centuries in traditional Chinese medicine as a common agent to treat a variety of conditions including diabetes, atherosclerosis, cancer as well as for boosting the immune system through potent antioxidant activity. Research has shown that mulberry plant materials comprise various physiologically active components including flavonoids, polyphenols, polysaccharides, 1-deoxynojirimycin (DNJ) identified as α-glucosidase inhibitor, fagomine, GABA or the like.

WO2013/078658 reports effects of mulberry juice on blood glucose levels following consumption of high glycemic index carbohydrate. EP3145332 A1 reports reduction of post-meal glucose and insulin spikes following administration of a composition comprising a combination of white kidney bean extract (1,200 mg), white mulberry extract (600 mg) and green coffee extract (400 mg) administered 15 minutes before a carbohydrate meal.

There is a persisting need in the food industry to further improve the nutritional solutions provided to diabetic subjects or subjects at risk for developing type-2 diabetes.

Alternative solutions instead of taking capsules or pills to lower postprandial glucose response can be appealing for consumers and increase consumer compliance.

An object of the present invention is to improve the state of the art and to provide a new and advantageous nutritional solution for improving the PPG profile in a subject, particularly in a diabetic or a subject at risk for developing T2D.

### SUMMARY

The inventor surprisingly found that a composition comprising a mulberry leaf extract (MLE), a source of fiber and a source of unsaturated fatty acids, administered with a meal provides a significant decrease in PPG. Also surprisingly, it was found that the intake of such composition in a milk based formula, provides a significant decrease in PPG.

Accordingly, in a non-limiting embodiment, the present invention provides a method of reducing postprandial glucose from a meal. The method comprises administering a composition comprising mulberry extract, a source of fiber and a source of unsaturated fatty acids together with a meal.

In a preferred embodiment, the mulberry extract is a mulberry leaf extract (MLE).

In a preferred embodiment the mulberry extract is a mulberry leaf extract (MLE) comprising at least 5% by weight of DNJ.

In a preferred embodiment, the fiber is a dietary fiber, a prebiotic fiber, cellulose, inulin, pectin, beta-glucan, resistant starch Fibersol-2®.

In a preferred embodiment the fiber is inulin. In a preferred embodiment the composition is a milk based formula.

In a preferred embodiment, the meal is a standard mixed meal and/or a carbohydrate-rich meal.

In a preferred embodiment, the meal is medium to high GI mixed meal.

In a preferred embodiment the meal is high GI mixed meal.

In a preferred embodiment, the meal is typical medium to high GI Chinese style mixed meal.

In another aspect the present invention provides a method of treating or preventing at least one condition for which reduced postprandial glucose and/or postprandial insulin from a meal is beneficial, the method comprising orally administering to an individual in need thereof or at risk thereof a composition comprising at least one mulberry extract (ME), at least one source of fiber and at least one source of unsaturated fatty acids, with the meal.

In a further aspect, the present invention provides a method of improving glycemic control and/or T2D management; help manage weight loss; regulating appetite after a meal by increasing GLP-1, the method comprising orally administering to an individual in need thereof or at risk thereof a composition comprising at least one mulberry extract (ME), at least one source of fiber and at least one source of unsaturated fatty acids, with a meal.

In another aspect, the present invention provides a method of decreasing fat deposition and/or managing glycemia control by lowering GIP. The method comprises administering a composition comprising mulberry extract, a source of fiber and a source of unsaturated fatty acids together with a meal.

In another aspect, the present invention provides a method of lowering postprandial BP to reduce cardiovascular risk. The method comprises administering a composition comprising mulberry extract, a source of fiber and a source of unsaturated fatty acids together with a meal.

In an embodiment, the individual is healthy.

In an embodiment, the individual has or is at risk of having at least one condition selected from the group consisting of overweight, obesity, diabetes, gestational diabetes mellitus (GDM), pre-diabetes and non Alcoholic Fatty Liver Disease (NAFLD).

In a last aspect, the present invention relates to a kit comprising a composition as described above and a digital tool.

In a preferred embodiment, the digital tool is a companion App.

Advantageously the composition according to the present invention administered with a meal, in particular a carbohydrate-rich meal, provides a significant decrease in postprandial glucose.

Additional features and advantages are described in, and will be apparent from, the following Detailed Description and the Figures.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages are by weight of the total weight of the composition unless expressed otherwise. Similarly, all ratios are by weight unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth. Ranges defined using “between” include the referenced endpoints.

As used herein and in the appended claims, the singular form of a word includes the plural, unless the context clearly dictates otherwise. Thus, the references “a,” “an” and “the” are generally inclusive of the plurals of the respective terms. For example, reference to “an ingredient” or “a method” includes a plurality of such “ingredients” or “methods.” The term “and/or” used in the context of “X and/or Y” should be interpreted as “X,” or “Y,” or “X and Y.” Similarly, “at least one of X or Y” should be interpreted as “X,” or “Y,” or “both X and Y.”

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components. “Consisting essentially of” means that the embodiment or component thereof comprises more than 50 wt. % of the individually identified components, preferably at least 75 wt. % of the individually identified components, more preferably at least 85 wt. % of the individually identified components, most preferably at least 95 wt. % of the individually identified components, for example at least 99 wt. % of the individually identified components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

“Animal” includes, but is not limited to, mammals, which includes but is not limited to rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage, e.g., an animal benefitting from reduced postprandial glucose. While the term “individual” or “subject” is often used herein to refer to a human, the present disclosure is not so limited. Accordingly, the term “individual” or “subject” refers to any animal, mammal or human that can benefit from the methods and compositions disclosed herein.

The relative terms “improved,” “reduced,” “enhanced” and the like refer to the effects of the method disclosed herein on postprandial glucose, particularly the administration of a composition containing WPM before administration of a meal (e.g., about thirty minutes before the meal), relative to administration of an identically formulated meal but administered without the WPM (e.g., no WPM within about one hour of the meal) or relative to administration of an identically formulated meal concurrently with the WPM (i.e., at approximately the same time).

As used herein, the terms “treat” and “treatment” mean to administer a composition as disclosed herein to a subject having a condition in order to lessen, reduce or improve at least one symptom associated with the condition and/or to slow down, reduce or block the progression of the condition. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

The terms “prevent” and “prevention” mean to administer a composition as disclosed herein to a subject is not showing any symptoms of the condition to reduce or prevent development of at least one symptom associated with the condition. Furthermore, “prevention” includes reduction of risk, incidence and/or severity of a condition or disorder. As used herein, an “effective amount” is an amount that treats or prevents a deficiency, treats or prevents a disease or medical condition in an individual, or, more generally, reduces symptoms, manages progression of the disease, or provides a nutritional, physiological, or medical benefit to the individual.

“Overweight” is defined for a human as a body mass index (BMI) between 23 and 27.5 kg/m2 or between 25 and 30 kg/m2 depending on population Similarly, “Obese” is defined for a human as a BMI of at least 27.5 kg/m2 or at least 30 kg/m2 for example 30-39.9 kg/m2 (WHO (World Health Organization). Physical status: the use and interpretation of anthropometry: report of a WHO expert committee. World Health Organ Tech Rep Ser. 1995; 854:1-452)

“Diabetes” encompasses both the type I and type II forms of the disease. Non-limiting examples of risk factors for diabetes include: waistline of more than 40 inches for men or 35 inches for women, blood pressure of 130/85 mmHg or higher, triglycerides above 150 mg/dl, fasting blood glucose greater than 100 mg/dl or high-density lipoprotein of less than 40 mg/dl in men or 50 mg/dl in women. Other risk factors for diabetes include: age >=45 years, family history of diabetes, sedentary lifestyle/low physical activity, history of gestational diabetes, other conditions associated with insulin resistance (acanthosis nigricans, non-alcoholic steatohepatitis, polycystic ovary syndrome), atherosclerotic cardiovascular disease, drugs/medication. Therefore, an “individual at risk of diabetes” may have one or more of these factors present.

“Pre-diabetes” is defined as a state of abnormal glucose homeostasis where blood glucose levels are elevated above those considered normal, but not as high as those required for a diagnosis of diabetes. The Chinese Diabetes Society (CDS) has defined prediabetes according to the following criteria:

| Categories of | Fasting plasma | 2h-plasma |
|---|---|---|
| prediabetes | glucose (FPG) | glucose (2h-PG) |
| (hyperglycaemia) | (mmol/L) | (mmol/L) |
| IFG | ≥6.1, <7.0 | <7.8 |
| (Impaired fasting glucose) | | |
| IGT | <7.0 | ≥7.8, <11.1 |
| (Impaired glucose tolerance) | | |

As used herein, “administering” includes another person providing a referenced composition to an individual so that the individual can consume the composition and also includes merely the act of the individual themselves consuming a referenced composition.

As used herein, “subsequently” means at least about five minutes later, preferably at least about ten minutes later.

The terms “food,” “food product” and “food composition” mean a composition that is intended for ingestion by an individual, such as a human, and that provides at least one nutrient to the individual. “Food” and its related terms include any food, feed, snack, food supplement, treat, meal substitute, or meal replacement, whether intended for a human or an animal. Animal food includes food or feed intended for any domesticated or wild species. In preferred embodiments, a food for an animal represents a pelleted, extruded, or dry food, for example, extruded pet foods such as foods for dogs and cats.

The terms “serving” or “unit dosage form,” as used herein, are interchangeable and refer to physically discrete units suitable as unitary dosages for human and animal subjects, each unit containing a predetermined quantity of the composition comprising WPM disclosed herein in an amount sufficient to produce the desired effect, preferably in association with a pharmaceutically acceptable diluent, carrier or vehicle. The specifications for the unit dosage form depend on the particular compounds employed, the effect to be achieved, and the pharmacodynamics associated with each compound in the host. In an embodiment, the unit dosage form can be a predetermined amount of liquid housed within a container such as a bottle.

### EMBODIMENTS

All references herein to treatment include curative, palliative and prophylactic treatment. Treatment may also include arresting progression in the severity of a disease. Both human and veterinary treatments are within the scope of the present disclosure. Preferably the composition according to the present invention is administered in a serving or unit dosage form that provides a therapeutically effective or prophylactically effective amount of the composition.

The individual may be a mammal such as a human, canine, feline, equine, caprine, bovine, ovine, porcine, cervine or a primate. Preferably the individual is a human.

In view of the above, an embodiment provided herein is a method of reducing glucose response of a meal, the method comprising: orally administering to an individual a composition comprising at least one mulberry extract (ME), at least one source of fiber and at least one source of unsaturated fatty acids, with a meal.

The PPG in the individual is preferably reduced relative to postprandial glucose from oral administration of a composition comprising ME comprising the same amount of DNJ, administered before (e.g. at least 5 minutes before) the administration of the meal.

The individual preferably has or is at risk of having at least one condition selected from the group consisting of obesity, type-2 diabetes, type-1 diabetes, pre-diabetes, gestational diabetes mellitus and NAFLD.

Another embodiment provided herein is a method of treating or preventing a condition for which reduced postprandial glucose from a meal is beneficial, the method comprising: orally administering to the individual a composition comprising mulberry extract (ME) together with a meal. The condition is preferably selected from the group consisting of obesity, pre-diabetes, diabetes or NAFLD.

In another embodiment the invention pertains to a composition comprising mulberry extract for use in the treatment and/or prevention of a disorder linked to an increase in plasma PPG concentration in an individual in need thereof or at risk thereof, wherein the ME are provided with a meal.

The disorder may be selected from the group consisting of metabolic syndrome, glucose intolerance, pre-diabetes, gestational diabetes mellitus, diabetes type-2 and NAFLD. In an embodiment the individual in need thereof or at risk thereof may be an overweight or obese individual.

In a preferred embodiment, the composition is for use in an obese, pre-diabetic or diabetic patient. A “pre-diabetic patient” is a subject showing insulin resistance or impaired glucose tolerance and is predisposed, for example by family history or genetics, for developing type-2 diabetes later in life. The use of a composition administered with a meal according to the invention would consequently reduce the risk and/or the development of insulin resistance, metabolic syndrome, glucose intolerance and type-2 diabetes in those subjects.

In another embodiment, the invention provides a composition comprising at least one mulberry extract (ME), at least one source of fiber and at least one source of unsaturated fatty acids for use in:
i) improving glycemic control and/or T2D management; help manage weight loss; regulating appetite after a meal by increasing GLP-1,
ii) decreasing fat deposition and/or managing glycemia control by lowering GIP
iii) decreasing CVD risk by lowering postprandial BP,
iv) improving insulin sensitivity by reducing the insulin secretion.

With regard to measuring insulin secretion, C-peptide may be a useful marker in the differential diagnosis of hyperinsulinemic-hypoglycemia (Senthil K. Venugopal “C-Peptide” StatPearls [Internet]. Treasure Island (FL): StatPearls Publishing; 2022 January 2021 May 29. NBK526026PMID: 30252282. Moreover, C-peptide is a marker of pancreatic beta cell function. As its degradation rate is slower than insulin, it affords a more stable window of beta cell response.

The composition according to the present invention is administered with the meal, i.e. during the meal, for example as part of the meal.

In some embodiments, the meal is main meal, diner or breakfast.

The composition according to the invention is intended to be consumed with a meal, in particular a carbohydrate-rich meal and/or a standard mixed meal.

In a preferred embodiment, the meal is a carbohydrate-rich meal.

In a preferred embodiment, the meal is medium to high GI mixed meal.

In a preferred embodiment the meal is a high GI mixed meal.

In a preferred embodiment, the meal is typical medium to high GI Chinese style mixed meal.

As used herein, “meal” refers to one or more food products consumed at substantially the same time as each other; preferably such that at least one macronutrient and at least one micronutrient are provided by consuming the meal; more preferably such that one or more proteins, one or more carbohydrates, one or more fats, one or more vitamins and one or more minerals are provided by consuming the meal. Preferably the meal comprises a plurality of food products. In a preferred embodiment, the meal macronutrient comprises 50%-60 Kcal % carbohydrate; 15%- 25% protein and 15%-25% fat.

As used herein “standard mixed meal” refers to refers to meal which provides all of protein, carbohydrate, fat, vitamins and minerals, in quantities and proportions suitable to maintain health or growth of an individual. In an embodiment, the meal provides 200 kcal to 1,000 kcal to the individual, preferably 250 kcal to 900 kcal, more preferably 300 kcal to 850 kcal, and most preferably 350 kcal to 800 kcal. The meal can be any meal, for example breakfast, lunch or dinner.

As used herein “carbohydrate rich meal” refers to meal where 40 to 90% Kcal of the meal energy intake are coming from the carbohydrates. In a preferred embodiment, the energy intake coming from the carbohydrate in such a meal is higher than 50%, more preferably higher than 60%, higher than 70%, higher than 80%.

The glycemic index is defined as the incremental area under the blood glucose response curve of a 50 g carbohydrate portion of a test food expressed as a percent of the response to the same amount of carbohydrate from a standard food taken by the same subject.

A food is considered to have a low GI if it is 55 or less; high GI if 70 or more; and mid-range (medium) GI if 56 to 69. As used herein, “medium to high GI mixed meal” refers to a meal having a GI above 55, preferably from 56 to 70 or more.

In an embodiment, the meal has the following energy intake breakdown:
50-65% Kcal from Carbohydrates
10-25% Kcal from Protein
10-25% Kcal from Fat

In a preferred embodiment, the meal has the following energy intake: 54% Kcal from carbohydrates; 23% Kcal from protein and 19% Kcal from fat.

In an embodiment, the glycaemic load of the meal is above 33 (see https://www.gisymbol.com/low-gi-explained). In a preferred embodiment, the glycemic load of the meal is from 40 to 80, preferably from 50 to 70. Available carbohydrates are defined as the sum of free sugars and complex carbohydrates like starch. We expect an effect with a meal containing from 30 to 80 g of available carbohydrates with at least 30 g from starch, preferably from 50 to 70 g of available carbohydrates with at least 50 g from starch.

In an embodiment the composition can typically be administered to the individual in a serving that provides about 50 mg to about 2 g of ME per serving, for example about 100 mg to about 2 g ME/serving, preferably about 100 mg to about 1 g ME/serving, such as about 100 mg to about 500 mg ME/serving, such as about 250 mg ME/serving.

The ME can have a concentration of DNJ at least 1% wt/wt (dry weight), least 2% wt/wt, at least 3% wt/wt, at least 4% wt/wt, for example about 5% wt/wt. In another embodiment the ME can have a concentration of DNJ at least 5% wt/wt, for example at least 6% wt/wt. In an embodiment, the composition comprising ME comprises at least 1 mg DNJ per g dry weight of the composition, preferably at least 2 mg DNJ, preferably at least 3 mg DNJ, preferably at least 4 mg DNJ, more preferably at least 5 mg DNJ, such as at least 6 mg DNJ per g dry weight of the composition.

In an embodiment the composition is administered to the individual in a serving that provides about 1 mg to about 100 mg of DNJ per serving, for example about 5 mg to about 50 mg DNJ/serving, preferably about 10 mg to about 30 mg DNJ/serving, or about 10 mg to about 20 mg DNJ/serving such as about 10 mg to about 15 mg ME/serving, such as about 12.5 mg DNJ/serving.

Research has shown that mulberry plant extracts comprise various physiologically active components including flavonoids, polyphenols, polysaccharides, 1-deoxynojirimycin (DNJ) identified as α-glucosidase inhibitor, fagomine, GABA. 1-deoxynojirimycin (DNJ) is thought to be the main active ingredient acting through potent α-glucosidase inhibition.

The mulberry extract applicable to the present invention can be of any Morus origin, including, but not limited to, White Mulberry (Morus alba L.), Black Mulberry (Morus nigra L.), American Mulberry (Morus celtidifolia Kunth), Red Mulberry (Morus rubra L.), hybrid forms between Morus alba and Morus rubra, Korean Mulberry (Morus australis), Himalayan Mulberry (Morus laevigata), and combinations thereof.

The mulberry extract applicable to the present invention can be derived from different parts of mulberry tree, including barks (trunk, twig or root), roots, buds, twigs, young shoots, leaves, fruits or a combination thereof. The mulberry extract can be in the form of e.g. dried powders such as dried powders milled from different parts of the tree. The starting plant material of mulberry extracts can be fresh, frozen or dried mulberry materials. The extract may be used as a liquid or dried concentrated solid. Typically, such an extract includes from at least about 1% w/v 1-DNJ.

In a preferred embodiment, the mulberry extract (ME) is a mulberry leaf extract (MLE).

Mulberry extracts can be prepared by procedures well known in the art. References in this aspect can be made to Chao Liu et al., Comparative analysis of 1-deoxynojirimycin contribution degree to a-glucosidase inhibitory activity and physiological distribution in Morus alba L, Industrial Crops and Products, 70 (2015) p309-315; Wenyu Yang et al., Studies on the methods of analyzing and extracting total alkaloids in mulberry, Lishizhen Medicine and Material Medical Research, 2008 (5); and CN104666427.

Mulberry leaf extracts are also commercially available, such from Karallief Inc, USA, ET-Chem.com, China, Nanjing NutriHerb BioTech Co., Ltd, China, or from Phynova Group Ltd.

The compositions of the present invention may typically comprise from about 1% to about 50%, including from about 2% to about 30%, such as from about 5% to about 20%, and also including from about 10% to about 15% of mulberry extract by weight of the composition.

In an embodiment, the composition provides at least a source of unsaturated fatty acids. In a preferred embodiment, the unsaturated fatty acids is selected from the group consisting of monounsaturated fatty acids and/or polyunsaturated fatty acids.

Preferably the mono-unsaturated fatty acid form part of a lipid mixture which contains at least about 50% by weight of mono-unsaturated fatty acids.

The lipid mixture may also include poly-unsaturated fatty acids; for example about 10% to about 40% by weight of poly-unsaturated fatty acids. A poly-unsaturated fatty acid mixture which is enriched in omega-3 fatty acids is preferred.

Suitable sources of mono-unsaturated fatty acids are vegetable oils which are rich in mono-unsaturated fatty acids. For example, vegetable oils such as olive oil, apricot oil, and oleic-acid rich oils obtained from sunflower, safflower or rapeseed hybrids, may be used.

Suitable sources of poly-unsaturated fatty acids are walnut, sunflower seeds, flaxseeds and poppy seeds; oils of palm, soybean, rapeseed and sunflower. Omega 3 fatty acids may be provided as fish oil and from oil of seeds such as chia seeds, hemp seeds and flaxseeds.

In a preferred embodiment, the amount of polyunsaturated fatty acids and/or monounsaturated fatty acids is at least about 1 g, preferably from 1 g to about 20 g, more preferably from 2.5 g to 5 g of the composition.

In an embodiment, the composition provides a source of at least one dietary fiber, preferably inulin. Dietary fiber is present in the composition in an amount from about 1.5 g to about 30 g, more preferably from about 1.5 g to about 5 g. In an embodiment, the composition comprises dietary fiber, such as inulin, in an amount from about 1.5 g to about 2.5 g. In a preferred embodiment, inulin has a DP>10.

Preferably the composition is in the form of a powder. In a preferred embodiment, the composition is a milk based powder that can be reconstituted in a liquid (e.g. water). In a preferred embodiment the milk can be from animal or plant origin. In an embodiment, the amount of skimmed milk is from about 50% to about 95%, more preferably from about 70% to about 90%. In a preferred embodiment, the composition comprises about 75 to 85% wt skimmed milk powder.

In a preferred embodiment, the composition is in a unit dose of 30 g; which comprises 23 g of skimmed milk powder, 789 mg mulberry leaf extract extract standardised to contain 1%1-DNJ (e.g. Reducose®), 3.06 g of an oil mix and 2.35 g of inulin, to be reconstituted with 180 ml of water.

The format of the compositions can contain excipients, emulsifiers, stabilizers and mixtures thereof, and the final formulation is preferably, in a powder/granulate format to be reconstituted in liquid.

The compositions of the present invention may further comprise a filler material to augment the bulk properties of the compositions. These filler materials may include any such material that is suitable for use in a composition.

The filler material may include any nutritional or non-nutritional ingredient that adds bulk, and in most instances will be substantially inert, and does not significantly negate the blood glucose benefits of the composition. The filler material most typically includes a fiber and/or carbohydrate having a low glycemic index.

The filler material, including any carbohydrate and/or fiber filler material, may represent enough of the finished product to provide the desired bulk or flow properties, but most typically represent from about 30% to about 90%, preferably from about 40% to about 85%, more preferably from about 50% to about 85%, and most preferably from about 75% to about 80%, by weight of the composition.

Carbohydrate sources suitable for use as a filler material in the compositions of the present invention, include those having a low glycemic index such as fructose and low DE maltodextrins as such ingredients do not introduce a high glycemic load into the composition. Other suitable filler material includes any dietary fiber suitable for human or animal use, including soluble and insoluble fiber, especially soluble fibres. Beneficial effects of soluble fibres on glucose response have been widely reported. Non-limiting examples of suitable soluble fibres include FOS, GOS, inulin, resistant maltodextrins, partially hydrolysed guar gum, polydextrose and combinations thereof.

Non limiting example of commercially available filler materials for use herein include Sunfiber® (Taiyo International, Inc.), which is a water-soluble dietary fiber produced by the enzymatic hydrolysis of Guar beans; Fibersol 2™ (Archer Daniels Midland Company), which is a digestion resistant maltodextrin; and polydextrose.

In an embodiment the compositions of the present invention additionally comprise a soluble fibre. In a preferred embodiment soluble fibre selected from polydextrose, a resistant maltodextrin (such as the soluble corn fiber Fibersol-2) and combinations thereof. In an embodiment the composition comprises ME and soluble fibre in a ratio fibre: ME of from about 1:1 to about 20:1, preferably from about 4:1 to about 10:1.

The composition may also comprise other filler, stabilizers, anti-caking agents, anti-oxidants or combinations thereof.

The composition may further comprise one or more additional components such as minerals; vitamins; salts; or functional additives including, for example, palatants, colorants, emulsifiers, antimicrobial or other preservatives. Non-limiting examples of suitable minerals for the compositions disclosed herein include calcium, phosphorous, potassium, sodium, iron, chloride, boron, copper, zinc, magnesium, manganese, iodine, selenium, chromium, molybdenum, fluoride and any combination thereof. Non-limiting examples of suitable vitamins for the compositions disclosed herein include water-soluble vitamins (such as thiamin (vitamin B1), riboflavin (vitamin B2), niacin (vitamin B3), pantothenic acid (vitamin B5), pyridoxine (vitamin B6), biotin (vitamin B7), myo-inositol (vitamin B8) folic acid (vitamin B9), cobalamin (vitamin B12), and vitamin C) and fat-soluble vitamins (such as vitamin A, vitamin D, vitamin E, and vitamin K) including salts, esters or derivatives thereof. Vitamin C, vitamin D, vitamin B3 and/or vitamin B12 are particularly useful in the composition. In a preferred embodiment the composition is supplemented with one or more vitamin and/or mineral selected from vitamin C, vitamin B3, zinc or any combination thereof.

### Digital Tool/Companion App

In a preferred embodiment, the composition is provided as a kit with a Companion App.

In some embodiments, the companion app is connected to a biofeedback device e.g. an activity tracker, a continuous glucose monitoring (CGM) device to measure acute postprandial glucose response (PPGR), in particular after consumption of the meal.

In some embodiments, the companion App is analyzing and recording the content of the meal and consumption of the composition according to the present invention with the meal.

In some embodiments, the companion App presents a questionnaire, displayed on the digital hub, to determine consumer wishes and concerns, it may record dietary habits, physical activity and/or lifestyle information. The companion App may determine/predict the cardiometabolic disease risk of the individual.

In some embodiments, the companion App tracks the progress towards the objectives, as well as activities through digital tools (connected watch, etc).

In some embodiments, the companion App may provide personalized recommendations, such as helping the individual adopting healthy lifestyle habits, for example.

Those skilled in the relevant art will appreciate which technology, logic and algorithm can be practiced to program, configure or construct the companion app or another equivalent digital tracking tool.

### EXAMPLE

### Example 1

### Milk Based Powder Composition

A composition according to the present invention comprises:

| Ingredient | Quantity [%] |
|---|---|
| Milk Skim Powder (MSK) | 77 |
| Oil Mix (PUFA + MUFA) | 10.2 |
| Inulin 89% Fiber DP>10 | 8 |
| Mulberry Leaf Extract | 2.6 |
| Spray Dried (1% 1-DNJ) | |
| Calcium Carbonate | 1.4 |
| Mineral mix | 0.52 |
| Vitamin Premix | 0.4 |
| Lecithin | 0.3 |
| Flavor | 0.23 |
| Water evaporation(−)/ absorption(+) | −0.65 |
| Composition | 100 |

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of reducing postprandial glucose from a meal, the method comprising:
orally administering to an individual a composition comprising at least one mulberry extract (ME), at least one source of fiber and at least one source of unsaturated fatty acids, with the meal.

2. The method of claim 1 wherein the individual has or is at risk of having at least one condition selected from the group consisting of overweight, obesity, type-1 diabetes, type-2 diabetes, gestational diabetes mellitus and pre-diabetes.

3. A method of treating or preventing at least one condition for which reduced postprandial glucose and/or postprandial insulin from a meal is beneficial, the method comprising:
orally administering to an individual in need thereof or at risk thereof a composition comprising at least one mulberry extract (ME), at least one source of fiber and at least one source of unsaturated fatty acids, with the meal.

4. A method of improving glycemic control and/or T2D management; help manage weight loss; regulating appetite after a meal by increasing GLP-1, the method comprising:
orally administering to an individual in need thereof or at risk thereof a composition comprising at least one mulberry extract (ME), at least one source of fiber and at least one source of unsaturated fatty acids, with the meal.

5-7. (canceled)

8. The method according to claim 3, wherein the at least one condition is selected from the group consisting of overweight, obesity, pre-diabetes, gestational diabetes mellitus, diabetes and NAFLD.

9. The method of claim 1 wherein the mulberry extract is a mulberry leaf extract.

10. The method of claim 1 wherein the fiber is selected from the group consisting of a prebiotic fiber, cellulose, inulin, pectin, beta-glucan, resistant starch Fibersol-2®.

11. The method of claim 1 wherein the fiber is inulin.

12. The method of claim 1, wherein the composition is a milk based formula.

13. The method of claim 1, wherein the meal is a standard mixed meal and/or a carbohydrate-rich meal.

14. The method of claim 1, wherein the meal is a medium to high GI mixed meal.

15. The method of claim 1 wherein the composition is administered to the individual in a serving comprising about 1 mg up to about 100 mg of 1-deoxynojirimycin (DNJ) per serving.

16. The method of claim 1, wherein the unsaturated fatty acids is selected from the group consisting of polyunsaturated fatty acids and/or monounsaturated fatty acids.

17. The method of claim 1, wherein the unsaturated fatty acids are in an amount of at least 1 g.

18. The method of claim 1, wherein the fiber is present in the composition in an amount from about 1.5 g to about30 g.

19. The method of claim 18, wherein the fiber is inulin.
